(19) United States
(12) Patent Application Publication
(10) Pub. No.: US 2023/0108822 A1
(43) Pub. Date: Apr. 6, 2023

(54) INJECTABLE COMPOSITION FOR SKIN AND SOFT TISSUE AUGMENTATION

(71) Applicant: MERZ PHARMA GMBH & CO. KGAA, Frankturt am Main (DE)

(72) Inventors: Bartosch NOWAG, Wiesbaden (DE); Panieta SCHAEFER, Bingen (DE)

(21) Appl. No.: 17/759,420

(22) PCT Filed: Feb. 4, 2021
(86) PCT No.: PCT/EP2021/052627

§ 371 Date: Jul. 25, 2022

(30) Foreign Application Priority Data
Feb. 6, 2020 (EP) 20155763.4

(51) Int. Cl. A61K 8/24; A61K 9/00; A61K 33/42; A61K 31/728; A61K 45/06; A61Q 19/00; A61K 8/73; A61Q 19/08

(52) U.S. Cl. CPC A61K 8/24; A61K 9/0019; A61K 33/42; A61K 31/728; A61K 45/06; A61Q 19/00; A61K 8/735; A61Q 19/08; A61K 2800/91; A61K 2800/412

(57) ABSTRACT

The present invention relates to the synergistic effect of calcium hydroxyapatite particles and hyaluronic acid on the bio-stimulation. The invention refers to an injectable composition comprising one or more types of calcium hydroxyapatite particles and hyaluronic acid and to a method for preparing such injectable composition. Furthermore, it relates to a cosmetic method for improving appearance of the skin and/or contour of a subject by means of the injectable composition. Moreover, the invention refers to the injectable composition for use in a method of treating a pathologic condition associated with pathologic deterioration of connective tissue.

The invention relates to injectable compositions comprising one or more types of calcium hydroxyapatite particles and hyaluronic acid and to a method for preparing such injectable compositions. Furthermore, it relates to a cosmetic method for improving appearance of the skin and/or contour of a subject by means of the injectable composition. Moreover, the invention refers to injectable compositions for use in a method of treating a pathologic condition associated with pathologic deterioration of connective tissue. Surprisingly, a synergistic effect of calcium hydroxyapatite particles and hyaluronic acid on the bio-stimulation was found.

In recent years, facial and body re-shaping, in particular reduction of undesired wrinkles, has gained increasing interest. For example, filling of wrinkles, breast reconstruction or augmentation, rejuvenation of the skin, soft-tissue augmentation of other kind, etc., are frequently performed. In order to avoid the need of surgical interventions in this context, a number of dermal fillers that can be injected subcutaneously or within the deeper layers of the skin have been developed or are under development.

For several of these purposes, filler materials have been used. A major drawback of many filler compounds is, however, that either biodegradation of such materials is often comparably rapid and the filler material is not suitable for long term solutions or that there is no biodegradation but rather a defensive reaction of the administered subject's body. Many filler materials are xenobiotics that are not tissue-like.

It is particularly desirable that at least a part of the filled tissue areas is finally filled by the administered subject's own tissue and/or extracellular matrix. This is achieved by products such as injectable calcium hydroxyapatite (CaHA, Ca₅(PO₄)₃(OH), Ca₅[OH|(PO₄)₃]) particles. Such fillers are e.g. described in U.S. Pat. No. 6,537,574. A commercial product comprising calcium hydroxyapatite particles is RADIESSE® (Merz Pharmaceuticals GmbH, Frankfurt, Germany).

Wrinkles can be effectively treated by administering subjects with RADIESSE® (calcium hydroxyapatite) sub-dermally. It was demonstrated that facial augment-tation is possible with such product (cf. Jacovella, Clinical Interventions in Aging, 2008, 3:161-174). Then, to some extend after several months, collagen is generated by fibroblasts in the tissue area, administered with the calcium hydroxyapatite particles without significant undesired side effects (cf. Coleman et al., Dermatologic Surgery, 2008, 34:S53-S55; Berlin et al., Dermatologic Surgery, 2008, 34:S64-S67). The administration of calcium hydroxyapatite particles was found to have a long-lasting volume increasing effect, even after calcium hydroxyapatite is degraded. The calcium hydroxyapatite is partly replaced by collagen. It was found that, four months after injection, collagen types I and III were significantly increased (Yutskovskaya and Kogan, Journal of Drugs in Dermatology, 2017, 16:68-74). The generation of collagen can be further improved.

As an alternative approach, the glycosaminoglycan hyaluronic acid (HA, hyaluronan, hyaluronate) are considered as a dermal filler. Also this compound is tissue-like. However, hyaluronic acid was found to have an insufficient long-lasting effect and, when used in high concentrations as required for a filling effect, undesired side-effects such as increase of inflammatory markers occur (cf. Yutskovskaya et al., Journal of Drugs in Dermatology, 2014, 13:47-52). In comparison to calcium hydroxyapatite particles, the long-lasting effect after several months is clearly less beneficial (cf. Yutskovskaya et al., Journal of Drugs in Dermatology, 2014, 13:47-52).

Hyaluronic acid was found to bear an undesired decrease in total protein expression and in generation of collagen (cf. Croce et al., Tissue & Cell, 2001, 33:326-331). Therefore, hyaluronic acid can be used as filler having a short-term activity, but is rather not used when it is intended to fill the subdermal areas with the subject's own tissue and/or extracellular matrix to obtain a long-lasting effect.

When, filling with collagen is intended, in view of the teaching of the prior art (cf. Croce et al., Tissue & Cell, 2001, 33:326-331), the person skilled in the art would thus refrain from administering hyaluronic acid.

In view of the above, there is still an unmet need for further improved dermal fillers which pharmaceutically/cosmetically acceptable and bear a long-term activity. Fillers which enable fillings with the subject's own tissue and/or extracellular matrix are desired. In particular, there is still a technical desire to improve collagen-stimulating effect of dermal fillers. This would have a quantitative improvement in skin quality of the administered subject.

Surprisingly, it has been found that a composition comprising one or more types of calcium hydroxyapatite particles in combination with hyaluronic acid leads to superior technical effects with respect to collagen stimulation. Surprisingly, against expectations from the prior art, a synergistic effect of calcium hydroxyapatite particles and hyaluronic acid on biostimulation has been found. This leads to the filling with the body's self-generated material, good body compatibility and desirable long-lasting effects. In particular, undesired wrinkels can be treated highly efficiently.

Accordingly, one aspect of the present invention relates to an injectable composition comprising:
(A) one or more types of calcium hydroxyapatite particles as component A; and
(B) hyaluronic acid as component B comprising:
   (B1) one or more types of high-molecular weight hyaluronic acid of a mean molecular weight in the range of from 1 to 5 MDa as component B1, or
   (B2) one or more types of low-molecular weight hyaluronic acid of a mean molecular weight in the range of from 5 to 100 kDa as component B2, or
   (B3) a combination of components B1 and B2; and
(C) one or more pharmaceutically acceptable carriers as component C comprising at least one viscous or liquid carrier; and optionally
(D) one or more local anesthetics as component D; and optionally
(E) one or more pharmaceutically acceptable additives other than components A, B, C and D as component E,

wherein the weight ratio of the components A:B, referred to the dry matters of components A and B, is in the range of from 1000:1 to 1:10.

As used herein, the weight ratio of components is typically referred to the dry matters of the components. The terms “dry matter”, “dry weight” and “solid contend” may be understood interchangeably in the broadest sense as generally understood in the art. The person skilled in the art will notice that the dry matter may refer to the weight of the respective component without solvents/diluents and other components. The dry matters may also be considered when the respective components are dissolved, suspended or are forming a (hydro)gel with other components. In this case, the theoretical dry matter may be calculated, i.e., the weight of the solvents/diluents and further components may be substracted from the total weight.

The injectable composition of the present invention can be used as a filler (e.g., a dermal and/or soft-tissue filler) that is superior to the existing filler on the market. Although it was expected that hyaluronic acid decreases collagen production, unexpectedly, in combination with calcium hydroxyapatite particles, the negative effect was overcome. An even stronger induction of collagen compared to calcium hydroxyapatite particles alone was found. Thus, a surprising synergism of hyaluronic acid and calcium hydroxyapatite particles was identified.

These results indicate that such composition is particularly well suitable for increasing expression of collagen. It is, thus, a particularly suitable dermal and soft-tissue filler.

Preferably, the injectable composition of the present invention is a pharmaceutically and/or cosmetically acceptable composition. The composition of the present invention may have any galenic form. In one embodiment, the injectable composition of the present invention is liquid or viscous. In another embodiment, the injectable composition of the present invention is pasty.

As used herein, the terms “liquid”, “viscous” and “pasty” may be understood in accordance with general understanding in the art. Preferably, “liquid” as used in the context of the present invention means having a viscosity of less than 10 mPas (millipascal-seconds, at standard conditions, 20° C., at 1013.25 hPa). Preferably, “viscous” as used in the context of the invention means having a viscosity of from 10 to 1000 mPas (at standard conditions, 20° C., at 1013.25 hPa). The terms “viscous”, “gel” and “gel-like” should be understood interchangeably.

Preferably, “pasty” as used in the context of the present invention means having a viscosity of between 1000 and 1000.000 mPas (at standard conditions, 20° C., at 1013.25 hPa). These viscosity values can be determined by any means, for example, by a rotational/oscillating viscometer, e.g., according DIN 53019-4:2016-10). According to the invention, when the injectable composition is a liquid, viscous or pasty injectable composition, the calcium hydroxyapatite particles (component A) are preferably dispersed in the injectable composition, i.e., in the liquid, viscous or pasty component of the injectable composition.

Accordingly, a liquid, viscous or pasty injectable composition is typically a dispersion. The hyaluronic acid (component B) is preferably partly or completely comprised in the liquid, viscous or pasty component of the injectable composition. It may be dissolved and/or emulsified. The hyaluronic acid (component B) or parts thereof may form a hydrogel. The optional further components C, D and/or E may be partly or completely comprised in the liquid, viscous or pasty component of the injectable composition or may be dispersed therein. When the injectable composition is a liquid, viscous or pasty injectable composition, at least parts of the hyaluronic acid (component B) may optionally form a hydrogel. Parts of the hyaluronic acid (component B) may optionally also be dissolved or emulsified. In a preferred embodiment, the injectable composition of the present invention is a gel. Thus, it is preferably a gel-like, i.e., viscous, injectable composition.

In an embodiment of the present invention, injectable composition is injectable into the skin or into other soft-tissue. In an embodiment of the present invention, injectable composition is usable for skin or other soft-tissue improvement. In an embodiment of the present invention, injectable composition is injectable (sub)cutaneously/(sub)dermally. Preferably, the injectable composition is suitable for injection into a mammal, in particular a human. Preferably, the composition of the present invention is preferably (essentially) sterile and is preferably a-pyrogenic.

As used herein, the terms “component” and “ingredient” may be understood interchangeably in the broadest sense as a part of the composition of the present invention.

As used herein, the term “pharmaceutically acceptable” may be understood in the broadest sense as any being reasonably usable in a pharmaceutical and/or cosmetic context. It will be understood that a pharmaceutically acceptable component or composition will typically also be inherently usable as being cosmetically acceptable. A pharmaceutically acceptable component or composition bears a low toxicity and can be administered to a human or animal (typically mammal) body without seriously harm this human or animal.

As used herein, mean molecular weight may be determined by any routine means suitable for this purpose such as, e.g., gel permeation chromatography (GPC), size exclusion chromatography (SEC), measuring the thickening effect (viscosimetry), mass spectrometry, etc. The mean molecular masses of the soluble fraction of hyaluronic acid types are preferably determined by gel permeation chromatography (GPC). The mean molecular masses of the insoluble, gel-forming fraction of hyaluronic acid types are preferably determined by measuring the thickening effect (viscosimetry) by routine experiments (e.g., at 25° C. by an EP monograph method on an Ubbelohe viscometer). As used herein, 1000 kDa (kilodaltons) are 1 MDa (megadalton).

The calcium hydroxyapatite particles may have any particle size. Preferably, in the context of the present invention, these are suitable for being injected. In other words, these are injectable calcium hydroxyapatite particles. Therefore, the calcium hydroxyapatite particles typically are microspheres and, thus, have a mean particle size in the micrometer range, i.e., in the range of 1 to 1000 μm.

As used herein, particle size may be determined by any means (e.g., light diffraction/scattering, sieving, microscopy, etc.). The number values shown herein refer to the (weight) mean size range determined by light scattering (PSA). Preferably, the calcium hydroxyapatite particles are (essentially) spherical. The calcium hydroxyapatite particles preferably have a D-ratio above 0.8, in particular above 0.9. In this context, a D-ratio of 1.0 indicates perfect roundness. Preferably, the calcium hydroxyapatite particles have a smooth surface.

Preferably, the calcium hydroxyapatite particles have a BET specific surface area of <1 m²/g, <0.5 m²/g, or <0.1 m²/g. In an embodiment of the present invention, the calcium hydroxyapatite particles have a mean particle size of from 1 to 150 μm, or of from 2 to 100 μm, or of from 5 to 80 μm, or of from 10 to 60 μm, or of from 15 to 50 μm, or of from 20 to 45 μm. For instance, the calcium hydroxyapatite particles may have a mean particle size of (about) 25 to (about) 45 μm. Such calcium hydroxyapatite particles are commercially available such as, e.g., comprised in the product RADIESSE® (Merz Pharmaceuticals GmbH, Germany).

According to the invention, the weight ratio of components A:B, referred to the dry matters of components A and B, is in the range of from 1000:1 to 1:10. In a preferred embodiment, the weight ratio of components A:B, referred to the dry matters of components A and B, is in the range of from 100:1 to 1:10, or of from 50:1 to 1:5, or of from 20:1 to 1:4, or of from 10:1 to 1:3, or of from 5:1 to 1:2, or of from 2:1 to 1:1.

As used herein, hyaluronic acid may be understood in the broadest sense as the (essentially) non-sulfated glycosaminoglycan and its salts as generally understood in the art. In accordance with general understanding in the art, it will be understood that the term “hyaluronic acid” also includes its salts. Hyaluronic acid may, for instance, have the CAS numbers 9004-61-9, 31799-91-4 (potassium salt), or 9067-32-7 (sodium salt). The terms “hyaluronic acid”, “hyaluronan” and “hyaluronate” and the abbreviation “HA” as used herein may be understood interchangeably.

In an embodiment of the present invention, hyaluronic acid (component B) is one or more types of high-molecular weight hyaluronic acid of a mean molecular weight in the range of from 1 to 5 MDa (component B1).

As experimentally shown, a single type of hyaluronic acid in the injectable composition of the present invention is already sufficient to obtain an unexpected technical effect. In particular, a high-molecular weight hyaluronic acid (component B1) in the injectable composition of the present invention is already sufficient to obtain an unexpected technical effect. Nevertheless, it was surprisingly found that a combination of two different species (components B1 and B2) is particularly beneficial.

In particular, combining high-molecular weight hyaluronic acid (component B1) and low-molecular weight hyaluronic acid (component B2) leads to unexpectedly beneficial technical effects.

In another embodiment of the present invention, hyaluronic acid (component B) is one or more types of low-molecular weight hyaluronic acid of a mean molecular weight in the range of from 5 to 100 kDa (component B2).

In a further embodiment of the present invention, hyaluronic acid (component B) is a combination of components B1 and B2, i.e., in other words, comprises hyaluronic acid of components B1 and B2, thus, comprises:
(B1) one or more types of high-molecular weight hyaluronic acid of a mean molecular weight in the range of from 1 to 5 MDa (component B1), and
(B2) one or more types of low-molecular weight hyaluronic acid of a mean molecular weight in the range of from 5 to 100 kDa (component B2).

In a preferred embodiment, the injectable composition comprises hyaluronic acid of components B1 and B2 and has a weight ratio of components B1:B2, referred to the dry matters of components B1 and B2, in the range of from 1000: 1 to 1:1.

In an embodiment of the present invention, the injectable composition comprises hyaluronic acid of components B1 and B2 and has a weight ratio of components B1:B2, referred to the dry matters of components B1 and B2, in the range of from 100: 1 to 1:1, or of from 100: 1 to 1:10, or of from 1000: 1 to 10:1, or of from 1000: 1 to 100: 1. In an embodiment of the present invention, the injectable composition comprises hyaluronic acid of components B1 and B2 and has a weight ratio of components B1:B2, referred to the dry matters of components B1 and B2, of (approximately) 1000: 1, or of (approximately) 500: 1, or of (approximately) 100: 1, or of (approximately) 50:1, or of (approximately) 10:1, or of (approximately) 5:1, or of (approximately) 1:1.

According to the present invention, high-molecular weight hyaluronic acid (component B1) has a mean molecular weight (MW) in the range of from 1 to 5 MDa. In a preferred embodiment, the component B1 has a mean molecular weight in the range of from 1.2 to 3.5 MDa, or of from 1.4 to 3.2 MDa, or of from 1.6 to 3.0 MDa, or of from 1.8 to 2.8 MDa, or of from 2.0 to 2.6 MDa, or of from 2.0 to 2.6 MDa, or of from 2.2 to 2.4 MDa. For example, component B1 may have a mean molecular weight of 1.5 MDa, 1.6 MDa, 1.7 MDa, 1.8 MDa, 1.9 MDa, 2.0 MDa, 2.1 MDa, 2.2 MDa, 2.3 MDa, 2.4 MDa, 2.5 MDa, or 2.6 MDa.

Hyaluronic acid of component B1 may be non-crosslinked or may be crosslinked hyaluronic acid. In a preferred embodiment, the hyaluronic acid of component B1 is non-crosslinked hyaluronic acid.

According to the present invention, low-molecular weight hyaluronic acid (component B2) has a mean molecular weight (MW) in the range of from 5 to 100 kDa.

In a preferred embodiment, the component B2 has a mean molecular weight of in the range of from 5 to 80 kDa, or of from 5 to 60 kDa, or of from 5 to 50 kDa, or of from 5 to 45 kDa, or of from 5 to 40 kDa. The component B2 may also have a mean molecular weight of in the range of from 10 to 80 kDa, or of from 15 to 60 kDa, or of from 20 to 50 kDa, or of from 25 to 45 kDa. For example, component B2 may have a mean molecular weight of 5 to 10 kDa, or of 5 to 15 kDa, or of 10 to 20 kDa or of 15 to 25 kDa, or of 20 to 30 kDa, or of 25 to 35 kDa, or of 30 to 40 kDa, or of 20 to 40 kDa, or of 35 to 45 kDa, or of 40 to 50 kDa.

Hyaluronic acid of component B2 may be non-crosslinked or may be crosslinked hyaluronic acid. In a preferred embodiment, the hyaluronic acid of component B2 is non-crosslinked hyaluronic acid.

In a preferred embodiment, the hyaluronic acid of component B1, if present, is non-crosslinked hyaluronic acid and component B2, if present, is non-crosslinked hyaluronic acid.

The injectable composition of the present invention comprises one or more pharmaceutically acceptable carriers as component C, wherein at least one carrier is a viscous or liquid carrier.

A pharmaceutically acceptable carrier (component C) according the present invention may be any carrier that is pharmaceutically acceptable, therefore, any carrier that is (essentially) non-toxic to the human or animal (typically mammal) body.

The one or more viscous or liquid carriers may be any pharmaceutically acceptable carrier that is viscous or liquid. For instance, the one or more viscous or liquid carriers may optionally comprise one or more pharmaceutically acceptable solvents such as, e.g., water, an aqueous buffer (e.g., a saline or phosphate buffered saline), dimethyl sulfoxide (DMSO), ethanol, vegetable oil, paraffin oil, glycerol, or combinations thereof. In one embodiment, the one or more viscous or liquid carriers may comprise or consist of an a-pyrogenic isotonic buffer, such as a physiological saline solution or a buffered physiological saline solution.

The hyaluronic acid (component B) may form a gel in combination with the one or more viscous or liquid carriers. The hyaluronic acid (component B) may form a hydrogel in combination with the one or more viscous or liquid carriers. The hyaluronic acid (component B) may be partly or completely dissolved the one or more viscous or liquid carriers. The one or more types of calcium hydroxyapatite particles (component A) may be dispersed in the one or more viscous or liquid carriers.

In a preferred embodiment, the sum of all pharmaceutically acceptable carriers (component C) comprises at least 10% by weight, at least 20% by weight, at least 50% by weight, at least 60% by weight, at least 70% by weight, or at least 80% by weight, or at least 90% by weight, referred to component C, of one or more viscous or liquid carriers.

As used herein, a content by weight (e.g., % by weight) typically refers to the component as such. In case of a solid matter, it typically refers to the dry matter of the respective component (% by weight, referred to dry matter).

In the injectable composition, the hyaluronic acid (component B) may form a (hydro)gel in combination with the one or more viscous or liquid carriers in which the one or more types of calcium hydroxyapatite particles (component A) may be dispersed. In the injectable composition, the hyaluronic acid (component B) may be partly or completely dissolved the one or more viscous or liquid carriers in which the one or more types of calcium hydroxyapatite particles (component A) may be dispersed.

The one or more pharmaceutically acceptable carriers (component C) may comprise one or more components selected from the group consisting of one or more polysaccharide derivatives other than hyaluronic acid or pharmaceutically acceptable salts thereof, one or more polysaccharides or pharmaceutically acceptable salts thereof, glycerol, one or more pharmaceutically acceptable solvents, and combinations or two or more thereof.

In a preferred embodiment, the hyaluronic acid (component B) forms a gel in combination with a pharmaceutically acceptable carrier (component C) that contains at least 50% by weight, at least 60% by weight, at least 70% by weight, or at least 80% by weight, or at least 90% by weight, referred to component C, of glycerol. The one or more types of calcium hydroxyapatite particles (component A) may be dispersed in this carrier.

In a preferred embodiment, the one or more pharmaceutically acceptable carriers (component C) further comprise one or more components selected from the group consisting of one or more polysaccharide derivatives other than hyaluronic acid or pharmaceutically acceptable salts thereof, one or more polysaccharides or pharmaceutically acceptable salts thereof, glycerol, and combinations or two or more thereof. In a preferred embodiment, the one or more pharmaceutically acceptable carriers (component C) comprise or are one or more types of carboxymethyl cellulose (CMC) or pharmaceutically acceptable salts thereof.

A local anesthetic (component D) may be any local anesthetic. In a preferred embodiment, the injectable composition comprises one or more local anesthetics (component D).

Preferably, a local anesthetic (component D), if present, is selected from the group consisting of lidocaine, ambucaine, amolanone, amylocaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethysoquin, dimethocaine, diperodon, dycyclonine, ecgonidine, ecgonine, ethyl chloride, etidocaine, beta-eucaine, euprocin, fenalcomine, formocaine, hexylcaine, hydroxytetracaine, isobutyl p-aminobenzoate, leucinocaine mesylate, levoxadrol, mepivacaine, meprylcaine, metabutoxycaine, methyl chloride, myrtecaine, naepaine, octacaine, orthocaine, oxethazaine, parethoxycaine, phenacaine, phenol, piperocaine, piridocaine, polidocanol, pramoxine, prilocaine, procaine, propanocaine, proparacaine, propipocaine, propoxycaine, psuedococaine, pyrrocaine, ropivacaine, salicyl alcohol, tetracaine, tolycaine, trimecaine, zolamine, and combinations of two or more thereof and salts thereof.

Alternative local anesthetics and combinations and salts thereof may also be used as component D. In a preferred embodiment, the local anesthetic (component D) is or comprises lidocaine. Combinations of two or more of the mentioned anesthetic agents, for example a combination of lidocaine and other “caine”-anesthetics like prilocaine, may also be used herein. A local anesthetic may make injection into an individual more comfortable.

One or more pharmaceutically acceptable additive (component E) other than components A, B, C and D may be additionally be present in the injectable composition of the present invention. Such pharmaceutically acceptable additive (component E) may be any further agent that is (essentially) non-toxic to the human or animal (typically mammal) body.

A pharmaceutically acceptable additive (component E) may exemplarily be selected from the group consisting of one or more detergents (e.g., sodium lauryl sulfate (SLS)/sodium doceyl sulfate (SDS)), one or more coloring agents (e.g., TiO₂, food coloring), one or more vitamins, one or more salts (e.g., sodium, potassium, magnesium, calcium, and/or zinc salts), one or more humectants (e.g., sorbitol, glycerol, mannitol, propylene glycol, polydextrose), one or more enzymes, one or more preserving agents (e.g., benzoic acid, methylparabene), one or more texturing agents (e.g., polyethylene glycol (PEG), sorbitol), one or more emulsifiers, one or more separating agents, one or more antioxidants, one or more herbal and plant extracts, one or more stabilizing agents, one or more polymers (e.g., hydroxypropyl methacrylamide (HPMA), polyethylene imine (PEI), polyethylene glycol (PEG)), one or more uptake mediators (e.g., polyethylene imine (PEI), dimethyl sulfoxide (DMSO), a cell-penetrating peptide (CPP), a protein transduction domain (PTD), an antimicrobial peptide, etc.) one or more antibody/antibodies, one or more counterstain dyes (e.g., fluorescein, fluorescein derivatives, Cy dyes, an Alexa Fluor dyes, S dyes, rhodamine, quantum dots, etc.), one or more cell proliferation factors, one or more homeopathic ingredients, and combinations of two or more thereof. A cell proliferation factor may improve cellular invasion into the administered composition of the present invention.

A dye may either improve localization of the injection (e.g., a pharmaceutically acceptable fluorescent dye like fluorescein or rhodamine) or may improve invisibility of the otherwise whitish composition of the present invention (e.g., by rendering it flesh-colored).

The composition may comprise the components A and B and, C, optionally, D and/or E in any amounts.

In a preferred embodiment, the injectable composition comprises at least 10% by weight, 5 to 90% by weight, 10 to 80% by weight, 20 to 77% by weight, 30 to 75% by weight, 40 to 73% by weight, 50 to 72% by weight, 50 to 80% by weight, or 60 to 80% by weight, based on the composition, of one or more types of calcium hydroxyapatite particles as component The weight percentages related to component A refer to dry matter of component A.

In a preferred embodiment, the injectable composition comprises up to 80% by weight, 0.1 to 75% by weight, 0.15 to 70% by weight, 0.2 to 60% by weight, 0.25 to 50% by weight, 0.5 to 20% by weight, or 1 to 10% by weight, based on the composition, of hyaluronic acid as component B. The weight percentages related to component B refer to dry matter of component B.

In a preferred embodiment, the injectable composition comprises up to 80% by weight, 1 to 80% by weight, 2 to 70% by weight, 3 to 60% by weight, 4 to 50% by weight, 5 to 45% by weight, 10 to 40% by weight, 20 to 35% by weight, or 25 to 35% by weight, based on the composition, of one or more types of pharmaceutically acceptable carriers as component C.

In a preferred embodiment, the injectable composition comprises up to 10% by weight, 0.001 to 5% by weight, 0.001 to 1% by weight, 0.01 to 2% by weight, or 0.1 to 1% by weight, based on the composition, of one or more local anesthetics as component D. In case component D in pure form is a solid compound, the weight percentages related to component D may refer to dry matter of component D.

In a preferred embodiment, the injectable composition comprises up to 10% by weight, 0.001 to 5% by weight, 0.01 to 2% by weight, or 0.1 to 1% by weight, based on the composition, of one or more pharmaceutically acceptable additives other than components A, B, C and D as component E. In case component E in pure form is a solid compound, the weight percentages related to component E may refer to dry matter of component E.

In a preferred embodiment, the injectable composition consists of:
(A) 10 to 80% by weight, referred to dry matter, based on the composition, of one or more types of calcium hydroxyapatite particles having a mean particle size of 1 to 150 μm as component A;
(B) 0.1 to 50% by weight, referred to dry matter, based on the composition, of hyaluronic acid consisting of:
   (B1) 50 to 100% by weight, referred to dry matter, based on the total content of hyaluronic acid, of one or more types of high-molecular weight hyaluronic acid of a mean molecular weight in the range of from 1 to 5 MDa as component B1; and
   (B2) 0 to 50% by weight, referred to dry matter, based on the total content of hyaluronic acid, of one or more types of low-molecular weight hyaluronic acid of a mean molecular weight in the range of from 5 to 100 kDa as component B2;
(C) 1 to 80% by weight, based on the composition, of one or more pharmaceutically acceptable carriers as component C comprising at least one viscous or liquid carrier;
(D) 0 to 10% by weight, based on the composition, of one or more local anesthetics as component D; and
(E) 0 to 10% by weight, based on the composition, of one or more pharmaceutically acceptable additives other than components A, B, C and D as component E.

In a preferred embodiment, the injectable composition consists of:
(A) 50 to 80% by weight, referred to dry matter, based on the composition, of one or more types of calcium hydroxyapatite particles having a mean particle size of 1 to 150 pm as component A;
(B) 0.1 to 50% by weight, referred to dry matter, referred to dry matter, based on the composition, of hyaluronic acid consisting of:
   (B1) 50 to 100% by weight, based on the total content of hyaluronic acid, of one or more types of high-molecular weight hyaluronic acid of a mean molecular weight in the range of from 1 to 5 MDa as component B1; and
   (B2) 0 to 50% by weight, referred to dry matter, based on the total content of hyaluronic acid, of one or more types of low-molecular weight hyaluronic acid of a mean molecular weight in the range of from 5 to 100 kDa as component B2;
(C) 20 to 35% by weight, based on the composition, of one or more pharmaceutically acceptable carriers as component C, in particular one or more polysaccharide derivatives other than hyaluronic acid or pharmaceutically acceptable salts thereof;
(D) 0.001 to 1% by weight, based on the composition, of one or more local anesthetics as component D; and
(E) 0 to 10% by weight, based on the composition, of one or more pharmaceutically acceptable additives other than components A, B, C and D as component E.

As indicated above, the injectable composition of the present invention may also used for cosmetic (non-therapeutic) and therapeutic purposes. As indicated above, the injectable composition of the present invention is particularly usable as a soft-tissue filler, in particular a dermal filler. Accordingly, the present invention also relates to the use of the injectable composition of the present invention for improving appearance of the skin and/or contour of a part of interest of the face or body of a subject. In particular, the present invention also relates to the use of the injectable composition of the present invention as a soft-tissue filler, in particular a dermal filler.

Accordingly, a further aspect of the present invention relates to a cosmetic method for improving appearance of the skin and/or contour of a part of interest of the face or body of a subject, said method including the following steps:
(i) providing components A, B and C and optionally D and optionally E of the present invention; and
(ii) injecting said components into the skin of the part of interest of the face or body of a subject.

In a preferred embodiment, the method including the following steps:
(i) providing an injectable composition of the present invention; and
(ii) injecting said injectable composition into the skin of the part of interest of the face or body.

The definitions and preferred embodiments as laid out in the context of the injectable composition above mutatis mutandis apply to the cosmetic method.

The present invention also relates to an injectable composition of the present invention for use in a method for improving appearance of the skin and/or contour of a part of interest of the face or body of a subject, said method including the following steps:
(i) components A, B and C and optionally D and optionally E, wherein the components are defined as in any of claims 1 to 9, preferably providing the injectable composition; and
(ii) injecting said injectable composition into the skin of the part of interest of the face or body.

The term “subject” (also: “individual” or “patient”) may be understood in the broadest sense as a human or animal, typically a mammal, preferably a human or a domestic mammal, who/which can be subjected to the a cosmetic method or treatment method with the injectable composition of the present invention.

As used throughout the present invention, the term “mammal” may be understood in the broadest sense as any mammalian animal. Preferably, the mammal is a human or a domestic animal such as an animal selected from the group consisting of mouse, rat, cow, pig, dog, cat, horse. Particularly preferably, a subject as used herein is a human. A human or animal administered with the injectable composition of the present invention can also be designated as a patient, independent on his/her health state an irrespective whether clinical symptoms occur or do not occur.

Injecting into the skin of the part of interest of the face or body may be injection in any part of the skin.

In an embodiment, the composition of the present invention is administered to (in particular injected into) soft tissue. In an embodiment, the composition of the present invention is administered to (in particular injected into) the dermis area, such as below the epidermis or above the hypodermis and as such may be injected subcutaneously/subdermally, hypodermically or intradermally, or some combinations. In one embodiment, the composition of the present invention is administered (in particular injected) subcutaneously, subdermally, and/or intradermally. In a preferred embodiment, injecting into the skin of the part of interest of the face or body is injecting subcutaneously or intradermally. Injection may be performed by any means such as, e.g., by a syringe.

As used herein, inducing may be understood in the broadest sense as (bio)stimulating production of a protein such as, e.g., collagen type. As indicated above, the injection may induce the production of collagen. The injection may induce the production of collagen type I. The injection may induce the production of collagen type III. The injection may induce the production of collagen types I and III.

In a preferred embodiment, the injectable composition may induce collagen production in skin cells more than component alone. In a preferred embodiment, the injectable composition may induce collagen type I production in skin cells more than component a alone. In a preferred embodiment, the injectable composition may induce collagen type III production in skin cells more than component a alone. In a preferred embodiment, the injectable composition may induce collagen types I and III production in skin cells more than component alone. In a preferred embodiment, the injectable composition may induce collagen (e.g., I and/or III) in skin cells more than component alone.

As used herein, in the context of a protein such as a collagen type, the term “production” may be understood in the broadest sense as generation of the protein such as one or more collagen types. This may be also understood as protein expression.

As used herein, induction of the production of a collagen type (in particular collagen type III) may be understood in the broadest sense as increasing the expression rate by at least 1% by weight, by at least 2% by weight, or by at least 5% by weight, or by at least 10% by weight, or by at least 20% by weight, or by at least 50%, or by at least 100% by weight, in comparison to comparable cells or a comparable tissue not administered with the injectable composition of the present invention.

Collagen (in particular collagen type III) is typically generated by fibroblasts. Accordingly, the cells are preferably fibroblasts or the tissue is preferably a tissue containing fibroblasts.

The cosmetic method may be used for any improvement of the appearance of the skin and/or contour of a part of interest of the face or body of a subject.

In a preferred embodiment, the cosmetic method is further characterized in that it is a method for a purpose selected from the group consisting of filling of wrinkles, improving facial lines, breast reconstruction or augmentation, rejuvenation of the skin, buttocks augmentation, remodeling of cheekbones, soft-tissue augmentation, filling facial wrinkles, improving glabellar lines, improving nasolabial folds, improving marionette lines, improving buccal commissures, oral commissures, improving peri-lip wrinkles, improving crow's feet, improving subdermal support of the brows, malar and buccal fat pads, improving tear troughs, nose, augmentation of lips, augmentation of cheeks, augmentation of peroral region, augmentation of scars such as acne scars, augmentation of infraorbital region, resolving facial asymmetries, improving jawlines, augmentation of chin, and combinations of two or more thereof. In a preferred embodiment, the cosmetic method is a method for filling of wrinkles or improving facial lines, in particular for filling of wrinkles.

In a preferred embodiment, the cosmetic method is a method for filling of wrinkles of interest of a subject, said method including the following steps:
(i) providing an injectable composition of the present invention; and
(ii) injecting said injectable composition subcutaneously or intradermally into the wrinkles of interest.

The step of injecting (step (ii)) may be injecting the injectable composition in connective tissue of the subdermal skin. In a preferred embodiment, the step (ii) is injecting the injectable composition in connective tissue of the subdermal skin and thereby stimulating the production of collagen, in particular collagen selected from collagen type III, collagen type I, or a combination of collagen type I and III.

As indicated above, the injectable composition of the present invention may also be used for therapeutic purposes. Accordingly, the present invention relates to the injectable composition of the present invention for use a medicament.

A further aspect of the present invention relates to the components A, B and C and optionally D and optionally E of the present invention for use in a method of treating a pathologic condition associated with pathologic deterioration of connective tissue.

A further aspect of the present invention relates to the injectable composition of the present invention for use in a method of treating a pathologic condition associated with pathologic deterioration of connective tissue.

It will be understood that the definitions and preferred embodiments as laid out in the context of the injectable composition and the cosmetic method above mutatis mutandis apply to the injectable composition for use and to the method of treating.

The present invention also relates to a method of treating a pathologic condition associated with pathologic deterioration of connective tissue in a subject, said method comprising administering said subject with a suitable amount of the injectable composition of the present invention.

The pathologic condition to be treated may be any pathologic condition associated with pathologic deterioration of connective tissue. In a preferred embodiment, the pathologic condition is selected from the group consisting of urinary incontinence, vesicoureteral reflux, vocal cord augmentation, lipotrophy (in particular lipotrophy in a patient suffering from human immunodeficiency virus (HIV)), a pathologic condition associated with age-related or pathologic deterioration of connective tissue, and combinations of two or more thereof.

The present invention further relates to a method for preparing an injectable composition. Preferably, the present invention further relates to a method for preparing an injectable composition of the present invention. Preferably, such method comprises the steps of:
(i) providing a solution comprising component A and a solution comprising component B and optionally one or more further components (e.g., selected from any of components C, D, and/or E), and
(ii) mixing the mixing components of step (i) to obtain the injectable composition.

Preferably, the obtained injectable composition induces collagen production in skin cells more than component alone.

A further aspect of the present invention relates to a method for preparing an injectable composition, said method comprising the steps of:
(i) providing:
   (a) one or more solutions comprising one or more types of calcium hydroxyapatite particles as component a; and
   (b) one or more solutions comprising one or more types of non-crosslinked hyaluronic acid as component b comprising:
      (B1) one or more types of non-crosslinked high-molecular weight hyaluronic acid of a mean molecular weight in the range of from 1 to 5 MDa as component B1, or
      (B2) one or more types of non-crosslinked low-molecular weight hyaluronic acid of a mean molecular weight in the range of from 5 to 100 kDa as component B2, or
      (B3) a combination of components B1 and B2; and optionally
   (c) one or more further components selected from the group consisting of one or more pharmaceutically acceptable carriers as component C, one or more local anesthetics as component D; one or more other pharmaceutically acceptable additives as component E, and a combination of two or more thereof; and
(ii) mixing components a and b, and optionally c, to obtain the injectable composition,

wherein the injectable composition induces (more/higher) collagen production in skin cells more than component alone.

In a preferred embodiment, the injectable composition obtained from this method is an injectable composition of the present invention as described above.

It will be understood that the definitions and preferred embodiments as laid out in the context of the injectable composition, the cosmetic method, the injectable composition for use, and the method of treating above mutatis mutandis apply to the method for preparing an injectable composition.

The step of mixing (step (ii)) may be performed by any means such as, e.g., by means of a mixer, a blender, a vortex mixer, a stirring unit, shaking, etc. Step (ii) may be performed at any temperature such as, e.g., at a temperature in the range of −4 to 60° C., or of 0 to 50° C., or of 5 to 40° C., or of 10 to 30° C., or of 15 to 25° C., such as, e.g., at room temperature (RT, 20° C.).

Optionally, the injectable composition of the present invention may be packaged. For instance, it may be packaged in syringes (for single use), vials, etc. A user manual may be added. Thus, the present invention also refers to a kit comprising the injectable composition and a user manual for cosmetic and/or therapeutic uses of the present invention.

The Examples described below and the claims further illustrate the invention.

### EXAMPLES

Materials

Calcium Hydroxyapatite Particles (CaHA):

CAS No. 1306-06-05 as used in the product RADIESSE®, K100086592, spherical microspheres, size distribution between >10 and <100 μm (as determined by laser diffraction), mean particle size 22-24 μm with a D-ratio of 0.93 (as determined by laser diffraction), material density approximately 3.1 g/cm³;

HA High:

High-molecular weight sodium hyaluronate having a molecular weight of 2.3 MDa, non-crosslinked, intrinsic viscosity 2.41 m³/kg (25° C., EP monograph method, Ubbelohde viscometer), HYALURONATE Na F100 (htl Biotechnology, France);

HA Low:

low-molecular weight sodium hyaluronate having a molecular weight range of 5 to 40 kDa, non-crosslinked;

Fibroblasts: fibroblastic cells (adult/single donor/breast (PromoCell, #412Z020-P3); Fibroblast growth medium: cell culture medium including 1 mM vitamin C and 1% by weight of PenStrep (penicillin-streptomycin);

Anti-Collagen III Antibody:

polyclonal antibody, rabbit, used as primary antibody (Invitrogen, PA5-34787);

Anti-Rabbit Antibody:

AlexaFluor488-labeled secondary antibody detecting the primary rabbit antibody (Invitrogen, A11034);

DAPI: 4′,6-diamidino-2-phenylindole (SIGMA, D9542); and

CellMask: deep red plasma membrane strain (Invitrogen, C10046).

Preparation of Hyaluronic Acid Solutions

A stock solution of low-molecular weight sodium hyaluronate (HA low) in fibroblast growth medium at a concentration of 20 mg/ml was prepared. Furthermore, a stock solution of high-molecular weight sodium hyaluronate (HA high) in fibroblast growth medium at a concentration of 2 mg/ml was prepared. At higher concentrations, undesired gelling occurred. These solutions were than further diluted in fibroblast growth medium to the desired concentrations and optionally mixed with each other.

Cell Culture and Sample Preparation

Fibroblasts were seeded at a density of 5000 cells per well. The cells were cultivated for 24 hours at standard conditions at 37° C. in fibroblast growth medium. After 24 hours, 200 μl of the hyaluronic acid-containing samples were added. The samples contained different amounts of hyaluronic acid. Some samples further contained 2 mg/ml calcium hydroxyapatite particles (CaHA). The treated cells were further incubated at standard conditions at 37° C. for 72 hours. After 72 hours, the medium was removed from the cells and the cells were fixed with cold methanol (−20° C.) for 10 minutes. Then, the fixed cells were washed three times with PBS (phosphate buffered saline) and stored at 4° C.

Collagen III Staining and Determination

The supernatant of the fixed cells was removed. Then, the cells were treated with 100 μl/well of a blocking buffer (5% by weight of albumin in PBS) for 2 hours at room temperature (RT). Subsequently, 70 μl/well of a solution of 6.7 μg/ml of the primary anti-collagen III antibody in Dako antibody solution (1:100) were added and incubated overnight in the dark at 4° C. on a horizontal mixer.

Subsequently, the treated fixed cells were washed three times with PBS. Subsequently, 70 μl/well of a solution of 10 μg/ml of the secondary AlexaFluo488-labeled anti-rabbit antibody in Dako antibody solution (1:200) were added and incubated for 1 hour at RT in the dark. The treated fixed cells were washed three times with PBS.

Subsequently, 70 μl/well of CellMask deep red plasma membrane strain was added in a dilution of 1:1000 in PBS (5 μg/ml). The fixed cells were incubated for 30 minutes at RT in the dark. Subsequently, 70 μl/well of a solution of 1 μg/ml DAPI solution in PBS (1:2000 dilution of an aliquot of 2 mg/ml) were added. The fixed cells were incubated for 10 minutes at RT in the dark. The treated fixed cells were washed three times with PBS.

The fluorescence signal was determined at an Imager for quantification of the signals. Furthermore, microscopic images were prepared. The results are depicted below.

Results

The results of collagen III production are shown in the following table. Herein, the synergistic effect of calcium hydroxyapatite particles (CaHA) and high-molecular weight hyaluronic acid (HA high) and low-molecular weight hyaluronic acid (HA low) was observed.

TABLE 1
Synergistic effect of the combination of CaHA with HA low
or HA high, respectively (n > 3, percentage expression to an
untreated control, rounded values)

| | | | | | Collagen III |
|---|---|---|---|---|---|
| Example/ | | | | cell count | expression |
| Comparative | HA low | HA high | CaHA | after 72 hours | after 72 hours |
| Example | [mg/ml] | [mg/ml] | [mg/ml] | [% of control] | [% of control] |
| 1 | 0.02 | | | 113.8 | 125.6 |
| 2 | 0.02 | | 2 | 99.3 | 157.9 |
| 3 | 2 | | | 115.6 | 128.3 |
| 4 | 2 | | 2 | 102.9 | 148.5 |
| 5 | 20 | | | 86.3 | 124.0 |
| 6 | 20 | | 2 | 80.6 | 154.4 |
| 7 | | 0.02 | | 108.0 | 125.0 |
| 8 | | 0.02 | 2 | 93.8 | 152.0 |
| 9 | | 0.2 | | 101.9 | 124.8 |
| 10 | | 0.2 | 2 | 87.4 | 156.7 |
| 11 | | 2 | | 99.3 | 121.5 |
| 12 | | 2 | 2 | 66.1 | 158.4 |
| 13 | | | 1 | 92.2 | 110.5 |
| 14 | | | 2 | 84.6 | 115.6 |
| 15 | | | 5 | 84.9 | 126.7 |

These data show the synergistic effect of hyaluronic acid (HA) and calcium hydroxyapatite particles (CaHA).

When adding HA alone, the collagen III Expression decreased with increasing HA concentration (cf. Comparative Examples 1, 3 and 5, as well as 7, 9 and 11). CaHA alone had a low effect with increasing CaHA concentration (cf. Comparative Examples 13-15). In contrast, the samples containing a combination of HA and CaHA showed a significant increase in collagen III expression.

Synergistic effects on collagen III expression were observed for the whole wide concentration range tested. Best synergistic effect on collagen III expression for HA low were observed for a combination of CaHA with 0.02 mg/ml of HA low. Best synergistic effect on collagen III expression for HA high were observed for a combination of CaHA with 2 mg/ml of HA high. The compositions of CaHA and HA maintained the vast majority of cells viable.

In a further independent experiment, lower concentrations of hyaluronic acid (HA) were tested. Thus, the CaHA:HA ratio was increased. The results are depicted in Table 2.

TABLE 2
Relative collagen III expression induced by a combination
of CaHA with HA low or HA high, respectively (percentage expression
to an untreated control, rounded values)

| Example/ | | | | Collagen III |
|---|---|---|---|---|
| Comparative | HA low | HA high | CaHA | Expression |
| Example | [mg/ml] | [mg/ml] | [mg/ml] | [% of control] |
| 16 | | | 2 | 112.1 |
| 17 | 0.00002 | | | 95.7 |
| 18 | 0.00002 | | 2 | 113.0 |
| 19 | 0.0002 | | | 95.1 |
| 20 | 0.0002 | | 2 | 109.8 |
| 21 | 0.002 | | | 94.4 |
| 22 | 0.002 | | 2 | 110.7 |
| 23 | | 0.00002 | | 99.5 |
| 24 | | 0.00002 | 2 | 114.3 |
| 25 | | 0.0002 | | 97.0 |
| 26 | | 0.0002 | 2 | 114.0 |
| 27 | | 0.002 | | 98.5 |
| 28 | | 0.002 | 2 | 111.0 |

At such low concentrations of hyaluronic acid (HA), only a very low or no synergistic effect of HA and CaHA was achieved. HA low alone had a negative effect on collagen III expression. This result confirms the findings in the art (cf. Croce et al., Tissue & Cell, 2001, 33:326-331). This undesired effect was remedied by addition of CaHA.

Furthermore, different combinations of HA low and HA high were tested. The results are depicted in Table 3.

TABLE 3
Relative collagen III expression induced by a combination of CaHA with
different combinations of HA low and HA high
(percentage expression to an untreated control, rounded values)

| | | | | | Collagen III |
|---|---|---|---|---|---|
| Example/ | | | | Collagen III | Expression |
| Comparative | HA low | HA high | CaHA | Expression | [% microspheres |
| Example | [mg/ml] | [mg/ml] | [mg/ml] | [% of control] | only of control] |
| 29 | 0.002 | 0.002 | | 97.9 | 87.9 |
| 30 | 0.02 | 0.002 | | 96.5 | 86.6 |
| 31 | 0.2 | 0.002 | | 99.7 | 88.9 |
| 32 | 2 | 0.002 | | 99.3 | 89.2 |
| 33 | 0.002 | 0.02 | | 94.8 | 85.2 |
| 34 | 0.02 | 0.02 | | 97.9 | 87.9 |
| 35 | 0.2 | 0.02 | | 98.0 | 88.0 |
| 36 | 2 | 0.02 | | 96.0 | 86.2 |
| 37 | 0.002 | 0.2 | | 98.8 | 88.7 |
| 38 | 0.02 | 0.2 | | 100.4 | 90.1 |
| 39 | 0.2 | 0.2 | | 99.1 | 88.9 |
| 40 | 2 | 0.2 | | 97.1 | 87.2 |
| 41 | 0.002 | 2 | | 93.6 | 84.0 |
| 42 | 0.02 | 2 | | 97.0 | 87.0 |
| 43 | 0.2 | 2 | | 98.9 | 88.8 |
| 44 | 2 | 2 | | 99.1 | 89.0 |
| 45 | 0.002 | 0.002 | 2 | 107.8 | 96.8 |
| 46 | 0.02 | 0.002 | 2 | 103.9 | 93.3 |
| 47 | 0.2 | 0.002 | 2 | 103.9 | 93.3 |
| 48 | 2 | 0.002 | 2 | 102.6 | 92.1 |
| 49 | 0.002 | 0.02 | 2 | 100.5 | 90.2 |
| 50 | 0.02 | 0.02 | 2 | 106.1 | 95.2 |
| 51 | 0.2 | 0.02 | 2 | 109.9 | 98.7 |
| 52 | 2 | 0.02 | 2 | 104.8 | 94.1 |
| 53 | 0.002 | 0.2 | 2 | 108.6 | 97.5 |
| 54 | 0.02 | 0.2 | 2 | 111.1 | 100.0 |
| 55 | 0.2 | 0.2 | 2 | 112.0 | 100.6 |
| 56 | 2 | 0.2 | 2 | 110.3 | 99.0 |
| 57 | 0.002 | 2 | 2 | 123.0 | 110.4 |
| 58 | 0.02 | 2 | 2 | 129.1 | 115.9 |
| 59 | 0.2 | 2 | 2 | 131.6 | 118.2 |
| 60 | 2 | 2 | 2 | 133.1 | 119.5 |

This experiment confirmed the presence of a synergistic effect of a combination of CaHA and HA. The synergistic effect occured in wide concentration ranges. It was found that HA high has a higher synergistic effect than HA low. Highest synergistic effects were found for a combination of 2 mg/ml of HA high and 0.02 mg/ml to 2 mg/ml of HA low.

Furthermore, microscopic images with collagen staining were generated. It was found that collagen is deposited around fibroblasts which are in contact with the calcium hydroxyapatite particles. This indicated that the present composition is a particularly suitable dermal filler. Fibroblasts invading the dermal filler materials can deposit collagen in the filled area.

In summary, synergistic effects of a combination of CaHA and HA were shown in a number of experiments.

These results indicate that the compositions according to the invention are particularly well suitable for increasing expression of collagen and, thus, provide particularly suitable dermal and soft-tissue fillers.

1-15. (canceled)

16. An injectable composition comprising:
(A) one or more types of calcium hydroxyapatite particles as component A;
(B) hyaluronic acid as component B comprising:
(B1) one or more types of high-molecular weight hyaluronic acid of a mean molecular weight in the range of from 1 to 5 MDa as component B1, or
(B2) one or more types of low-molecular weight hyaluronic acid of a mean molecular weight in the range of from 5 to 100 kDa as component B2, or
(B3) a combination of components B1 and B2;
(C) one or more pharmaceutically acceptable carriers as component C comprising at least one viscous or liquid carrier;
(D) optionally, one or more local anesthetics as component D; and
(E) optionally, one or more pharmaceutically acceptable additives other than components A, B, C, and D as component E,
wherein the weight ratio of components A:B, referred to the dry matters of components A and B, is in the range of from 1000: 1 to 1:10.

17. The injectable composition of claim 16, wherein the injectable composition comprises hyaluronic acid of components B1 and B2 and has a weight ratio of components B1:B2, referred to the dry matters of components B1 and B2, in the range of from 1000:1 to 1:1.

18. The injectable composition of claim 16, wherein the weight ratio of components A:B, referred to the dry matters of components A and B, is in the range of from 100: 1 to 1:10, or of from 50:1 to 1:5, or of from 20:1 to 1:4, or of from 10:1 to 1:3, or of from 5:1 to 1:2, or of from 2:1 to 1:1.

19. The injectable composition of claim 16, wherein the hyaluronic acid of component B1, if present, is non-crosslinked hyaluronic acid and component B2, if present, is non-crosslinked hyaluronic acid.

20. The injectable composition of claim 16, wherein component B1 has a mean molecular weight in the range of from 1.2 to 3.5 MDa, or of from 1.4 to 3.2 MDa, or of from 1.6 to 3.0 MDa, or of from 1.8 to 2.8 MDa, or of from 2.0 to 2.6 MDa, or of from 2.0 to 2.6 MDa, or of from 2.2 to 2.4 MDa.

21. The injectable composition of claim 16, wherein component B2 has a mean molecular weight of in the range of from 5 to 80 kDa, or of from 5 to 60 kDa, or of from 5 to 50 kDa, or of from 5 to 45 kDa, or of from 5 to 40 kDa.

22. The injectable composition of claim 16, wherein the calcium hydroxyapatite particles have a mean particle size of from 1 to 150 μm, or of from 2 to 100 μm, or of from 5 to 80 μm, or of from 10 to 60 μm, or of from 15 to 50 μm, or of from 20 to 45 μm.

23. The injectable composition of claim 16, wherein the injectable composition comprises one or more pharmaceutically acceptable carriers as component C.

24. The injectable composition of claim 16, wherein the injectable composition consists of:
(A) 10 to 80% by weight, referred to dry matter, based on the composition, of one or more types of calcium hydroxyapatite particles having a mean particle size of 1 to 150 μm as component A;
(B) 0.1 to 50% by weight, referred to dry matter, based on the composition, of hyaluronic acid consisting of:
(B1) 50 to 100% by weight, referred to dry matter, based on the total content of hyaluronic acid, of one or more types of high-molecular weight hyaluronic acid of a mean molecular weight in the range of from 1 to 5 MDa as component B1; and
(B2) 0 to 50% by weight, referred to dry matter, based on the total content of hyaluronic acid, of one or more types of low-molecular weight hyaluronic acid of a mean molecular weight in the range of from 5 to 100 kDa as component B2;
(C) 1 to 80% by weight, based on the composition, of one or more pharmaceutically acceptable carriers as component C comprising at least one viscous or liquid carrier;
(D) 0 to 10% by weight, based on the composition, of one or more local anesthetics as component D; and
(E) 0 to 10% by weight, based on the composition, of one or more pharmaceutically acceptable additives other than components A, B, C, and D as component E.

25. A cosmetic method for improving appearance of the skin and/or contour of a part of interest of the face or body of a subject, the method including the following steps:
(i) providing components A, B, C, optionally D, and optionally E, wherein the components are defined as in claim 16; and
(ii) injecting the components into the skin of the part of interest of the face or body of a subject.

26. The cosmetic method of claim 25, wherein the method includes the following steps:
(i) providing an injectable composition; and
(ii) injecting the injectable composition into the skin of the part of interest of the face or body of a subject,
wherein the injectable composition comprises:
(A) one or more types of calcium hydroxyapatite particles as component A;
(B) hyaluronic acid as component B comprising:
(B1) one or more types of high-molecular weight hyaluronic acid of a mean molecular weight in the range of from 1 to 5 MDa as component B1, or
(B2) one or more types of low-molecular weight hyaluronic acid of a mean molecular weight in the range of from 5 to 100 kDa as component B2, or
(B3) a combination of components B1 and B2;
(C) one or more pharmaceutically acceptable carriers as component C comprising at least one viscous or liquid carrier;
(D) optionally, one or more local anesthetics as component D; and
(E) optionally, one or more pharmaceutically acceptable additives other than components A, B, C, and D as component E,
wherein the weight ratio of components A:B, referred to the dry matters of components A and B, is in the range of from 1000: 1 to 1:10.

27. The cosmetic method of claim 25, wherein the method is further characterized in that it is a method for a purpose selected from the group consisting of filling of wrinkles, improving facial lines, breast reconstruction or augmentation, rejuvenation of the skin, buttocks augmentation, remodeling of cheekbones, soft-tissue augmentation, filling facial wrinkles, improving glabellar lines, improving nasolabial folds, improving marionette lines, improving buccal commissures, oral commissures, improving peri-lip wrinkles, improving crow's feet, improving subdermal support of the brows, malar and buccal fat pads, improving tear troughs or nose, augmentation of lips, augmentation of cheeks, augmentation of peroral region, augmentation of scars such as acne scars, augmentation of infraorbital region, resolving facial asymmetries, improving jawlines, augmentation of chin, and combinations of two or more thereof.

28. The cosmetic method of claim 25, wherein the step (ii) is injecting the injectable composition in connective tissue of the subdermal skin and thereby stimulating the production of collagen.

29. The injectable composition of claim 16 for use in a method of treating a pathologic condition associated with pathologic deterioration of connective tissue.

30. A method for preparing an injectable composition, wherein the method comprising the steps of:
(i) providing:
(a) one or more solutions comprising one or more types of calcium hydroxyapatite particles as component a;
(b) one or more solutions comprising one or more types of non-crosslinked hyaluronic acid as component b comprising:
(B1) one or more types of non-crosslinked high-molecular weight hyaluronic acid of a mean molecular weight in the range of from 1 to 5 MDa as component B1, or
(B2) one or more types of non-crosslinked low-molecular weight hyaluronic acid of a mean molecular weight in the range of from 5 to 100 kDa as component B2, or
(B3) a combination of components B1 and B2; and
(c) optionally, one or more further components selected from the group consisting of one or more pharmaceutically acceptable carriers as component C, one or more local anesthetics as component D, one or more other pharmaceutically acceptable additives as component E, and a combination of two or more thereof; and
(ii) mixing components a and b, and optionally, c, to obtain the injectable composition, wherein the injectable composition induces collagen production in the skin cells than component a, calcium hydroxyapatite particles, or component b, non-crosslinked hyaluronic acid, alone.

31. The injectable composition of claim 23, wherein the one or more pharmaceutically acceptable carriers comprise one or more components selected from the group consisting of one or more polysaccharide derivatives other than hyaluronic acid or pharmaceutically acceptable salts thereof, one or more polysaccharides or pharmaceutically acceptable salts thereof, glycerol, and combinations or two or more thereof.

32. The cosmetic method of claim 27, wherein the method is a method for filling of wrinkles of interest of a subject comprising the following steps:
(i) providing an injectable composition; and
(ii) injecting the injectable composition subcutaneously or intradermally into the wrinkles of interest,
wherein the injectable composition comprises:
(A) one or more types of calcium hydroxyapatite particles as component A;
(B) hyaluronic acid as component B comprising:
(B1) one or more types of high-molecular weight hyaluronic acid of a mean molecular weight in the range of from 1 to 5 MDa as component B1, or
(B2) one or more types of low-molecular weight hyaluronic acid of a mean molecular weight in the range of from 5 to 100 kDa as component B2, or
(B3) a combination of components B1 and B2;
(C) one or more pharmaceutically acceptable carriers as component C comprising at least one viscous or liquid carrier;
(D) optionally, one or more local anesthetics as component D; and
(E) optionally, one or more pharmaceutically acceptable additives other than components A, B, C, and D as component E,
wherein the weight ratio of components A:B, referred to the dry matters of components A and B, is in the range of from 1000: 1 to 1:10.

33. The cosmetic method of claim 28, wherein the collagen is selected from collagen type III, collagen type I, or a combination of collagen type I and III.

34. The injectable composition of claim 29, wherein the pathologic condition is selected from the group consisting of urinary incontinence, vesicoureteral reflux, vocal cord augmentation, lipoatrophy, a pathologic condition associated with age-related or pathologic deterioration of connective tissue, and combinations of two or more thereof.

35. The injectable composition of claim 29, wherein the lipoatrophy is lipoatrophy in a patient suffering from human immunodeficiency virus (HIV).
